(19) United States
(12) Patent Application Publication
(10) Pub. No.: US 2007/0053858 A1
(43) Pub. Date: Mar. 8, 2007

(54) Personal care composition

(76) Inventors: Paul Robert Tanner, Lebanon, OH (US); Donald Lynn Bissett, Hamilton, OH (US); Robert John Elsbrock, Cincinnati, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/412,354

(22) Filed: Apr. 27, 2006

Related U.S. Application Data
(63) Continuation-in-part of application No. 10/097,716, filed on Mar. 13, 2002.
(60) Provisional application No. 60/277,805, filed on Mar. 22, 2001.

(51) Int. Cl. A61K 8/49; A61K 8/73

(52) U.S. Cl. 424/62; 424/70.13; 514/62; 514/356; 514/563

(57) ABSTRACT

Personal care composition comprising a vitamin B3 compound, a sugar amine, a hydroquinone compound, and a dermatologically-acceptable carrier.

### CROSS REFERENCE TO RELATED APPLICATION

This application is a continuation-in-part of U.S. application Ser. No. 10/097,716, filed Mar. 13, 2002, pending, which claims the benefit of U.S. Provisional Application No. 60/277,805, filed Mar. 22, 2001.

### TECHNICAL FIELD

The present invention relates to topical compositions comprising a vitamin B3 compound, a sugar amine, and a hydroquinone compound, and methods of use thereof.

### BACKGROUND

A continuing need exists to improve the health and/or physical appearance of the skin, including the appearance of wrinkles, fine lines, pores, and discoloration, such as redness due to dry skin and/or rosacea, sallowness, and hyperpigmentation, such as age spots. Age spots are considered by many to be a particularly undesirable sign of aging. Although it may be possible to conceal the appearance of age spots with cosmetic compositions, it would be more desirable to reduce or even eliminate the hyperpigmentation. This would enhance the skin's natural appearance, and reduce the consumer's perceived need for concealing cosmetics. In addition, consumers tend to prefer skin care products that are either white or relatively uncolored. A need also exists, therefore, for personal care compositions that can reduce hyperpigmentation of skin, as well as to provide a benefit to the skin, and which are acceptable to consumers.

### SUMMARY

The present invention meets the aforementioned needs. Compositions comprising sugar amines (also known as amino sugars) in combination with other selected skin care actives are useful for providing benefits to the skin. Applicants believe that compositions comprising a sugar amine together with a hydroquinone compound and a vitamin B3 compound are useful for providing a benefit to the skin while reducing the appearance of age spots. In particular, targeted application of such compositions to hyperpigmented areas is especially useful for improving the appearance of such areas of skin.

The following describe some non-limiting embodiments of the present invention.

According to the first embodiment of the present invention, a personal care composition is provided comprising a vitamin B3 compound, a sugar amine, and hydroquinone.

According to another embodiment of the present invention, a method of regulating the condition of mammalian skin is provided comprising the step of applying to mammalian skin in need of regulation the personal care composition of the first embodiment. Alternatively, a method for reducing the appearance of hyperpigmented portions of skin is provided, comprising the step of applying the personal care composition to the hyperpigmented area of skin.

According to yet another embodiment of the present invention, a kit is provided comprising a composition according to the first embodiment and an implement, for example, suitable for targeted application of the composition to an area of skin in need of regulation.

These and other features, aspects, and advantages of the present invention will become evident to those skilled in the art from a reading of the present disclosure.

### DETAILED DESCRIPTION

The present invention describes a personal care composition useful for improving the appearance of visible discontinuities in mammalian skin and for improving the condition of mammalian skin. The composition may be used in a variety of personal care products, non-limiting examples of which include moisturizers, conditioners, cleansers, sunscreens, anti-aging compounds, and combinations thereof. The composition may be in a variety of forms, including but not limited to an emulsion, lotion, liquid, solid, cream, gel, mousse, ointment, paste, serum, stick, spray, tonic, etc. In one embodiment, the composition is in a form suitable for use with an implement, for example, such as a swab and/or a pen.

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. All measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

Herein, “personal care composition” means compositions suitable for topical application on mammalian keratinous tissue. “Skin care actives,” or “actives,” as used herein, means compounds that, when applied to the skin, provide a benefit or improvement to the skin. It is to be understood that skin care actives are useful not only for application to skin, but also to hair, nails and other mammalian keratinous tissue.

“Keratinous tissue,” as used herein, refers to keratin-containing layers disposed as the outermost protective covering of mammals which includes, but is not limited to, skin, hair, nails, cuticles, etc.

“Dermatologically acceptable,” as used herein, means that the compositions or components described are suitable for use in contact with human keratinous tissue without undue toxicity, incompatibility, instability, allergic response, and the like.

“Effective amount” as used herein means an amount of a compound or composition sufficient to significantly induce a positive benefit, preferably a positive keratinous tissue appearance or feel benefit, including independently or in combination the benefits disclosed herein, but low enough to avoid serious side effects (i.e., to provide a reasonable benefit to risk ratio, within the scope of sound judgment of the skilled artisan).

Herein, “delivery enhancement device” means any device that increases the amount of active ingredient applied to and/or into the skin relative to the amount of active ingredient that is delivered without using the device.

Herein, “regulating skin condition” means improving skin appearance and/or feel, for example, by providing a smoother appearance and/or feel. Herein, “improving skin condition” means effecting a visually and/or tactilely perceptible positive change in skin appearance and feel. Conditions that may be regulated and/or improved include, but are not limited to, one or more of the following: Reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by hyperpigmentation, etc.

As used herein, “signs of skin aging,” include, but are not limited to, all outward visibly and tactilely perceptible manifestations, as well as any macro- or microeffects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

I. Skin Care Actives

1. Sugar Amines (Amino Sugars)

The composition of the present invention comprises a sugar amine, also known as amino sugars. As used herein, “sugar amine” refers to an amine derivative of a six-carbon sugar, and includes salts and derivatives thereof. Examples of sugar amine compounds useful in the present invention are described in U.S. Pat. No. 6,159,485. In one embodiment, the composition comprises from about 0.001% to about 20%, alternatively from about 0.1% to about 10%, and alternatively from about 2% to about 5%, of the sugar amine.

Examples of sugar amines that are useful herein include glucosamine, N-acetyl glucosamine, mannosamine, N-acetyl mannosamine, galactosamine, N-acetyl galactosamine, their salts and derivatives, and mixtures thereof. In one embodiment, the sugar amine is glucosamine, and alternatively, the sugar amine is N-acetyl glucosamine.

2. Vitamin B₃ Compounds

The composition of the present invention comprises a vitamin B₃ compound, useful, for example, as an antioxidant and as a skin-lightener, and understood to include one or more vitamin B3 compounds, and salts and derivatives thereof. Non-limiting examples of suitable vitamin B₃ compounds include nicotinic acid esters, including non-vasodilating esters of nicotinic acid, nicotinyl amino acids, nicotinyl alcohol esters of carboxylic acids, nicotinic acid N-oxide, niacinamide, their salts and derivatives, and mixtures thereof. In one embodiment, the vitamin B₃ compound is niacinamide.

The compositions may comprise from about 0.01% to about 50%, alternatively from about 0.1% to about 10%, alternatively from about 0.5% to about 10%, alternatively from about 1% to about 5%, and alternatively from about 2% to about 5%, of the vitamin B₃ compound.

3. Hydroquinone Compounds

The composition of the present invention comprises a hydroquinone compound, also useful, for example, as an antioxidant and skin-lightener. The hydroquinone compounds of the present invention are understood to include hydroquinone and hydroquinone derivatives, which include but are not limited to p-benzoquinone, mono-tert-butylhydroquinone (MTBHQ), 2,5-di-tert-butylhydroquinone (DTBHQ), hydroquinone momomethyl ether (HQMME), toluhydroquinone (THQ); and salts and mixtures thereof. In one embodiment, the hydroquinone compound is hydroquinone.

The composition may comprise from about 0.001% to about 2%, alternatively from about 0.1% to about 5%, and alternatively from about 1.5% to about 4% of one or more hydroquinone compounds.

4. N-Acyl Amino Acids

The composition of the present invention may comprise an N-acyl amino acid. In one embodiment, the composition may comprise from about 0.1% to about 5%, alternatively from about 0.1% to about 2%, and alternatively from about 0.2% to about 1%, of an N-acyl amino acid.

Suitable N-acyl amino acid compounds include, but are not limited to, N-acyl phenylalanine, N-acyl tyrosine, their isomers, including their D and L isomers, salts, derivatives, and mixtures thereof. In one embodiment, the N-acyl amino acid is N-undecylenoyl-L-phenylalanine, commercially available under the tradename SEPIWHITE® from Seppic (France).

5. Sunscreens

The composition of the present invention may comprise one or more sunscreen actives (or sunscreen agents) and/or ultraviolet light absorbers. Herein, “sunscreen” includes sunscreen actives, ultraviolet light absorbers, physical sunblocks, and combinations thereof. Sunscreen actives and ultraviolet light absorbers may be organic or inorganic. Examples of suitable sunscreens are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93. Particularly suitable sunscreen actives include benzophenone, benzophenone-1, benzophenone-2, benzophenone-3, benzophenone-4, benzophenone-5, benzophenone-6, benzophenone-7, benzophenone-8, benzophenone-9, benzophenone-10, benzophenone-11, benzophenone-12, benzotriazolyl dodecyl p-cresol, 3-benzylidene camphor, benzylidene camphor sulfonic acid, benzyl salicylate, bis-ethylhexyloxyphenol methoxyphenyl triazine, bornelone, bumetrizole, butyl methoxydibenzoyl-methane, butyl PABA (p-aminobenzoic acid), cinnamidopropyl-trimonium chloride, cinoxate, dea-methoxycinnamate, dibenzoxazoyl naphthalene, di-t-butyl hydroxy-benzylidene camphor, diethylamino hydroxy-benzoyl hexyl benzoate, diethylhexyl butamido triazone, diethylhexyl 2,6-naphthalate, diisopropyl ethyl cinnamate, diisopropyl methyl cinnamate, di-methoxycinnamido-propyl ethyldimonium chloride ether, dimethyl PABA ethyl cetearyldimonium tosylate, dimorpholino-pyridazinone, dimorpholino-pyridazinone, disodium bisethylphenyl triaminotriazine stilbenedisulfonate, disodium distyrylbiphenyl disulfonate, disodium phenyl dibenzimidazole tetrasulfonate, drometrizole, drometrizole trisiloxane, ethyl dihydroxypropyl PABA, ethyl diisopropyl-cinnamate, ethylhexyl bis-isopentylbenzoxazolylphenyl melamine, ethyl dimethoxybenz-ylidene dioxoimidazolidine propionate, ethylhexyl dimethyl PABA, ethylhexyl methoxycinnamate, ethylhexyl methoxydibenzoyl-methane, ethylhexyl salicylate, ethylhexyl triazone, ethyl methoxycinnamate, ethyl PABA, ethyl urocanate, etocrylene, 4-(2-beta-glucopyrano-siloxy) propoxy-2-hydroxybenzophenone, glyceryl ethylhexanoate dimethoxycinnamate, glyceryl PABA, glycol salicylate, hexanediol disalicylate, homosalate, isoamyl cinnamate, isoamyl p-methoxycinnamate, isopentyl trimethoxy-cinnamate trisiloxane, isopropylbenzyl salicylate, isopropyl dibenzoylmethane, isopropyl methoxy-cinnamate, kaempferia galanga root extract, menthyl anthranilate, menthyl salicylate, methoxycinnamido-propyl hydroxysultaine, methoxycinnamido-propyl laurdimonium tosylate, 4-methylbenzylidene camphor, methylene bis-benzotriazolyl tetramethylbutyl-phenol, octocrylene, octrizole, PABA, PEG-25 PABA, phenylbenzimidazole sulfonic acid, polyacrylamidomethyl benzylidene camphor, polyamide-2, polyquaternium-59, polysilicone-15, potassium methoxy-cinnamate, potassium phenyl-benzimidazole sulfonate, red petrolatum, sodium benzotriazoyl butylphenol sulfonate, sodium phenylbenz-imidazole sulfonate, sodium urocanate, TEA-phenylbenzimid-azole sulfonate, TEA-salicylate, terephthalylidene dicamphor sulfonic acid, tetrabutyl phenyl hydroxybenzoate, titanium dioxide, urocanic acid, zinc cerium oxide, zinc oxide, and mixtures thereof.

In one embodiment, the composition comprises from about 1% to about 30%, and alternatively from about 2% to about 20% of one or more sunscreens. Exact amounts will vary depending upon the chosen sunscreen active and/or ultraviolet light absorber and the desired Sun Protection Factor (SPF), and are within the knowledge and judgment of one of skill in the art. In one embodiment, the sunscreen provides an SPF of from about 5 to about 60, alternatively from about 8 to about 45, and alternatively from about 15 to about 30.

6. Particulate Materials

The composition of the present invention may comprise a particulate material. Nonlimiting examples of particulate materials useful in the present invention include colored and uncolored pigments, interference pigments, inorganic powders, organic powders, composite powders, optical brightener particles, exfoliants and combinations thereof. These particulates can be platelet shaped, spherical, elongated or needle-shaped, or irregularly shaped, surface coated or uncoated, porous or non-porous, charged or uncharged, and can be added to the current compositions as a powder or as a pre-dispersion. These particulate materials may provide a wide range of functions, including but not limited to modifying skin feel, masking the appearance of certain skin characteristics such as blotchy areas, age spots, freckles, fine lines, wrinkles, and pores, absorbing excess skin sebum/oils, reducing skin shine, improving application properties of the composition, masking the color of other components of the composition, filling in skin pores, lines and wrinkles, and reducing migration of liquid materials on the skin. The composition may comprise from about 0.01% to about 20%, alternatively from about 0.05% to about 10%, and alternatively from about 0.1% to about 5%.

Particulate materials useful herein include but are not limited to bismuth oxychloride, sericite, mica, mica treated with barium sulfate or other materials, zeolite, kaolin, silica, boron nitride, lauroyl lysine, nylon, polyethylene, talc, styrene, polypropylene, polystyrene, ethylene/acrylic acid copolymer, sericite, aluminum oxide, silicone resin, barium sulfate, calcium carbonate, cellulose acetate, PTFE, polymethyl methacrylate, starch, modified starches such as aluminum starch octenyl succinate, silk, glass, fibers, ground seeds, pumice, and mixtures thereof. In one embodiment, the particulate material is a spherical powder with an average primary particle size from about 0.1 microns to about 75 microns, and alternatively from about 0.2 microns to about 30 microns.

Also useful herein are interference pigments, for the purpose of the present invention defined as thin platelike layered particles having two or more layers of controlled thickness with different refractive indices. The layers yield a characteristic reflected color from the interference of typically two, but occasionally more, light reflections, from different layers of the platelike particle. The most common examples of interference pigments are micas layered with about 50 nm to about 300 nm films of TiO2, Fe2O3, silica, tin oxide, and/or Cr2O3. Such pigments often are pearlescent. Pearl pigments reflect, refract and transmit light because of the transparency of pigment particles and the large difference in the refractive index of mica platelets and, for example, the titanium dioxide coating. Useful interference pigments are available commercially from a wide variety of suppliers, for example, Rona (Timiron™ and Dichrona™) and Eckart (e.g. Prestige™ and Prestige Silk™). Especially preferred are interference pigments with smaller particle sizes, with an average diameter of individual particles less than about 75 microns in the longest direction, preferably with an average diameter less than about 50 microns.

Other pigments useful in the present invention provide color primarily through selective absorption of specific wavelengths of visible light, and include inorganic pigments, organic pigments and combinations thereof. Examples of useful inorganic pigments include iron oxides, ferric ammonium ferrocyanide, manganese violet, ultramarine blue, and Chrome oxide. Organic pigments can include natural colorants and synthetic monomeric and polymeric colorants. An example is phthalocyanine blue and green pigment. Also useful are lakes, primary FD&C or D&C lakes and blends thereof. Also useful are encapsulated soluble or insoluble dyes and other colorants. Inorganic white or uncolored pigments useful in the present invention, for example TiO2, ZnO, or ZrO2, are commercially available from a number of sources. One example of a suitable particulate material contains the material available from U.S. Cosmetics (TRONOX™ TiO2 series, and SAT-T CR837, a rutile TiO2). Particularly preferred are charged dispersions of titanium dioxide, as are disclosed in U.S. Pat. No. 5,997,887.

The pigments/powders useful herein can be surface treated to provide added stability of color and/or for ease of formulation. Non-limiting examples of suitable coating materials include silicones, lecithin, amino acids, metal soaps, polyethylene and collagen. These surface treatments may be hydrophobic or hydrophilic, with hydrophobically treatments being preferred. Particularly useful hydrophobic pigment treatments include polysiloxane treatments such as those disclosed in U.S. Pat. No. 5,143,722.

In one embodiment, the composition may be substantially free of colorants, such as pigments, powders, dyes and other cosmetic colorants, wherein “substantially free” is understood to mean that although a small amount of colorant may incidentally be present, the amount is insufficient to effectively conceal or mask the appearance of a hyperpigmented portion of skin such as an age spot. This amount will vary depending upon the type of colorant, and would be known to one of skill in the art. In one embodiment, the total amount of colorant is less than 0.1%.

7. Other Skin Care Actives

The composition of the present invention may comprise one or more of the following skin care actives: desquamation actives, including but not limited to sulfhydryl compounds, salicylic acid, zwitterionic surfactants, and derivatives and mixtures thereof; anti-acne actives; anti-wrinkle actives and/or anti-atrophy actives; thiols, for example, ethane thiol and glutathione; hydroxy acids; skin peel agents; flavonoids; peptides, for example, peptides derived from soy proteins, palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, and Cu-histidine-glycine-glycine (Cu-HGG, also known as IAMIN®); anti-oxidants, such as Coenzyme Q₁₀ (ubiquinone, vitamin Q₁₀); retinoids; other vitamin B compounds, for example, thiamine (vitamin B1), pantothenic acid (vitamin B5), L-carnitine (vitamin B_{T}), riboflavin (vitamin B2), cobalamine (vitamin B12), pangamic acid or diisopropylamine dichloroacetate (vitamin B15's); anti-oxidants/radical scavengers, including but not limited to ascorbic acid (vitamin C) and its salts, ascorbyl esters of fatty acids, ascorbic acid derivatives (for example, magnesium ascorbyl phosphate and ascorbyl glucoside), tocopherol (vitamin E), tocopherol sorbate, tocopherol acetate and other esters of tocopherol, and idebenone; chelating agents; anti-inflammatory agents, such as corticosteroids, nonsteroidal anti-inflammatory agents (“NSAIDS”), “natural” anti-inflammatory agents, allantoin, bisabolol, and compounds of the licorice plant family (Glycyrrhiza glabra); anti-cellulite agents, for example caffeine, theophylline, theobromine, and aminophylline; topical anaesthetics; other skin lightening agents such as kojic acid, arbutin, ascorbic acid and derivatives thereof (e.g., magnesium ascorbyl phosphate or sodium ascorbyl phosphate), and extracts (e.g., mulberry extract, placental extract); antimicrobial and/or antifungal actives; terpene alcohols; farnesol; geraniol; phytantriols; conditioning agents, such as glycerol, urea, guanidine, and sucrose polyesters; and derivatives and mixtures of any of the above.

II. Dermatologically Acceptable Carrier

The personal care compositions of the present invention may comprise a dermatologically acceptable carrier. The composition may comprise from about 30% to about 99%, alternatively from about 60% to about 95%, alternatively from about 70% to about 90%, of the dermatologically acceptable carrier.

The carrier can be a wide variety of types, non-limiting examples of which include solutions, dispersions, emulsions and combinations thereof. Herein, “emulsions” generally contain an aqueous phase and an oil phase. The oils may be derived from animals, plants, or petroleum, may be natural or synthetic, and may include silicone oils. Emulsion carriers include, but are not limited to oil-in-water, water-in-oil and water-in-oil-in-water emulsions. In one embodiment, the carrier comprises an oil-in-water emulsion and/or a water-in-oil emulsion. Alternatively, the emulsion is an oil-in-water emulsion. The emulsions may comprise from about 0.01% to about 10%, and alternatively from about 0.1% to about 5%, of a nonionic, anionic or cationic emulsifier, and combinations thereof. The carrier further may comprise a conditioning agent and/or a thickening agent.

1. Conditioning Agents

The compositions of the present invention may comprise a conditioning agent, including a humectant, moisturizer, and/or skin conditioner. The composition may comprise from about 0.01% to about 20%, alternatively from about 0.1% to about 10%, and alternatively from about 0.5% to about 7%, of a conditioning agent. In embodiment, the conditioning agent is selected from urea, guanidine, sucrose polyester, panthenol, dexpanthenol, allantoin, glycerol, and mixtures thereof.

2. Thickening Agent

The compositions of the present invention may comprise from about 0.05% to about 5%, alternatively from about 0.1% to about 4%, and alternatively from about 0.25% to about 3%, of a thickening agent. Suitable classes of thickening agents include, but are not limited to carboxylic acid polymers, crosslinked polyacrylate polymers, polyacrylamide polymers, polysaccharides, gums and mixtures thereof. Non-limiting examples of suitable thickening agents are described in the CTFA International Cosmetic Ingredient Dictionary, 10^{th} Ed. (2004), pp. 2294-96.

III. Methods for Regulating Skin Condition

The present invention described one or more methods of regulating the condition of mammalian skin. The method comprises the step of topically applying to mammalian skin in need of regulation the personal care composition comprising a vitamin B3 compound, a sugar amine, and a hydroquinone compound. In an alternative embodiment, a method for reducing the appearance of hyperpigmented portions of skin is provided, comprising the step of applying the personal care composition primarily to the hyperpigmented area of skin (also referred to herein as “targeted application”).

The composition may be applied by a variety of means, including by rubbing, wiping or dabbing with hands or fingers, or by means of an implement and/or delivery enhancement device. Non-limiting examples of implements include a sponge or sponge-tipped applicator, a swab (for example, a cotton-tipped swab), a pen optionally comprising a foam or sponge applicator, a brush, a wipe, and combinations thereof. Non-limiting examples of delivery enhancement devices include mechanical, electrical, ultrasonic and/or other energy devices. The composition may be pre-applied to the applicator, and for example delivered to the user pre-packaged as such, or the user may be instructed to apply the composition to the applicator prior to use. Alternatively, the composition may be stored in an implement, for example, in a cotton-tipped swab. The composition may be transferred to the applicator directly from the container, for example, by squeezing and/or breaking or by other suitable means. The composition may be applied to the keratinous tissue by contacting the applicator and composition to the skin. Contact may include, for example, light pressure, dabbing, rubbing, wiping, or any other suitable means. When targeted application is desired, the composition may be applied to the desired area of keratinous tissue by means of a suitable applicator, for example, an applicator which allows the composition to be selectively applied to the target area and minimally, or not at all, to the surrounding keratinous tissue.

The amount of the composition applied, the frequency of application and the period of use will vary widely depending upon the level of components of a given composition and the level of regulation desired. For example, from about 0.01 g composition/cm² to about 1 g composition/cm² of keratinous tissue may be applied. In one embodiment, the compositions are applied at least once daily, where “daily” and “days” mean a 24-hour period. For example, the compositions may be applied daily for 30 consecutive days, alternatively for 14 consecutive days, alternatively for 7 consecutive days and alternatively for 2 consecutive days.

The method may comprise the step of inducing a temperature change in the composition and/or in the keratinous tissue either simultaneously or sequentially with the step of applying the composition. The method further may comprise additional steps which form part of a treatment or application regimen, including the steps of applying at least one additional composition, ingesting one or more dietary supplements, cleansing, etc.

IV. Kit

The present invention further provides a kit comprising at least one composition described herein. The kit may comprise an outer packaging unit, which in turn may comprise one or more inner packaging units. The inner and outer packaging units may be of any type suitable for containing, presenting and/or reasonably protecting from damage the contents of the kit. The kit further may comprise an implement, which may be suitable for targeted delivery of the composition to a desired area of keratinous tissue. The composition may be packaged separately from the implement, or may be contained within the implement. The kit further may comprise a plurality of components, including but not limited to, one or more additional compositions, one or more orally ingestible dietary supplements, an implement, a delivery enhancement device, a temperature change element, instructions for use of the implement and/or device, instructions for complying with suitable application regimens, instructions for targeted delivery, a substrate, and combinations thereof.

When an implement such as a pen is included, a non-limiting example of which is SHO127 F2 applicator from Shya Hsin™, one example of suitable instructions includes the following. Cleanse skin thoroughly before applying. Apply a thin layer of the product on the most visible dark spots, twice daily. To use: Turn the mechanism at the base of the pen until a drop of product appears at the end of the tip. Apply the concentrate on the most visible facial age spots, lightly massage product in by using the tip of the applicator to help penetration. If desired, you may follow up with your moisturizer or foundation makeup. Sun exposure should be limited by using a sunscreen agent, a sun blocking agent, or protective clothing.

When an implement such as a swab is included, a non-limiting example of which is an XPress Tip™ from SwabPlus™, one example of suitable instructions includes the following. Directions: Cleanse skin thoroughly before applying. Apply a thin layer of the product on the most visible dark spots, twice daily. To use: Hold swab with soft tip pointing up, slowly push the upper tube down into the lower tube. Treatment product will flow up into the soft tip, continue to push product until desired amount of product is visible at the swab tip. Apply the concentrate on the most visible facial age spots, lightly massage product in by using the tip of the applicator to help penetration. If desired, you may follow up with your moisturizer or foundation makeup. Sun exposure should be limited by using a sunscreen agent, a sun blocking agent, or protective clothing. Dispose of swab after use.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Examples 1-5

A lotion/cream skin care composition may be prepared from the following components:

| | Example 1 | Example 2 | Example 3 | Example 4 | Example 5 |
|---|---|---|---|---|---|
| Water Phase: | | | | | |
| Water | q.s. | q.s. | q.s. | q.s. | q.s. |
| Hydroquinone | 2.0 | 2.0 | 1.5 | 4.0 | 2.0 |
| Retinol Propionate | — | 0.1 | — | — | — |
| Glycerin | 7.0 | 10.0 | 20.0 | 5.0 | 10.0 |
| Niacinamide | 4.0 | 5.0 | 10.0 | 0.1 | 3.5 |
| N-Acetyl Glucosamine | 0.1 | 2.0 | 5.0 | 0.5 | 2.5 |
| Pentylene Glycol | 1.0 | 1.0 | — | 1.0 | — |
| Butylene Glycol | — | — | — | 1.0 | — |
| Triethanolamine | — | — | — | 0.07 | 0.35 |
| Dex-panthenol | 0.5 | 0.5 | 2.0 | 0.2 | 0.5 |
| Benzyl Alcohol | 0.25 | 0.25 | 0.5 | 0.25 | 0.25 |
| Sodium ascorbyl Phosphate | 0.02 | 0.02 | 0.1 | — | — |
| Disodium EDTA | 0.07 | 0.1 | 0.05 | 0.5 | 0.05 |
| Sodium Metabisulfite | 0.1 | 0.1 | 0.05 | 0.15 | 0.1 |
| Methylparaben | 0.1 | 0.1 | 0.1 | 0.1 | 0.1 |
| Oil Phase | | | | | |
| Octinoxate | 3.0 | 1.0 | 3.0 | — | 3.0 |
| Octisalate | — | — | 5.0 | — | — |
| Sepiwhite MSH¹ | — | — | — | 0.2 | 1.0 |
| Isopropyl Isostearate | — | — | 1.0 | — | — |
| Isohexadecane | 3.0 | 3.0 | 2.0 | 3.0 | 3.0 |
| Sucrose Polycottonseedate | 0.7 | 0.7 | — | — | — |
| Cetyl Alcohol | 0.3 | 0.4 | 0.5 | 0.5 | 0.3 |
| Stearyl Alcohol | 0.5 | 0.6 | 0.5 | 0.5 | 0.5 |
| Behenyl Alcohol | 0.4 | 0.5 | 0.5 | 0.5 | 0.4 |
| Polymethylsilsesquioxane | 0.3 | 0.5 | — | — | 0.5 |
| Ethylparaben | 0.2 | 0.2 | 0.2 | 0.2 | 0.2 |
| PEG-100 Stearate | 0.1 | 0.1 | 0.1 | 0.1 | 0.1 |
| Propylparaben | 0.1 | 0.1 | 0.1 | 0.1 | 0.1 |
| Tocopheryl Acetate | 0.1 | 0.5 | — | 0.1 | 0.5 |
| Cetearyl Glucoside | 0.2 | 0.2 | 0.2 | 0.2 | 0.2 |
| Thickener | | | | | |
| Sepigel 305² | 2.5 | 2.5 | — | 2.5 | 2.5 |
| Simulgel EG³ | — | — | — | 2.0 | — |
| Cosmedia SPL⁴ | — | — | 1.5 | — | — |
| Additional Ingredients | | | | | |
| Dimethicone/Dimethiconol | 2.0 | 2.0 | 1.0 | — | 1.0 |
| Polyethylene⁵ | — | — | 2.0 | — | — |
| Dry-Flo AF⁶ | — | — | — | 1.0 | — |
| KTZ Interfine Red⁷ | 0.5 | — | — | — | — |
| Total: | 100% | 100% | 100% | 100% | 100% |

¹Undecylenoyl phenylalanine from Seppic ™ (France)
²Polyacrylamide, C13-14 Isoparaffin, and Laueth-7 from Seppic ™
³Sodium Acrylate/Sodium Acryloyldimethyl Taurate Copolymer, Isohexadecane, and Polysorbate 80 from Seppic ™
⁴Sodium Polyacrylate, Hydrogenated Polydecene, and PPG-5-Laureth-5 from Cognis
⁵Microthene FN510-00 from Equistar ™
⁶Corn Starch Modified from National Starch and Chemical Company ™
⁷Titanium dioxide coated mica available from Kobo Products ™ Inc.

In a suitable vessel, combine the water phase ingredients and heat to 75° C. In a separate suitable vessel, combine the oil phase ingredients and heat to 75° C. Add the oil phase to the water phase and mill the resulting emulsion (eg., with a Tekmar T-25 rotor-stator mill) until thoroughly mixed. Add the thickener to the emulsion and cool the emulsion to 45° C. while stirring. Add the remaining ingredients. Cool the product with stirring to 30° C. and pour into suitable containers.

In one example, fill the compositions in examples 1 through 5 into plunger-type individual dose swabs (XPress Tips™ from SwabPlus™) for targeted application directly to specific areas of the skin, such as age spots. Alternatively, fill the compositions from examples 1 through 5 into applicator pens with a soft applicator tip (SHO127 F2 applicator from Shya Hsin™) for targeted application of the compositions to the desired areas of the skin. Alternatively, fill the compositions of examples 1 through 5 into 15 ml tubes with a long, narrow orifice for targeted application to the desired areas of the skin.

### Examples 6-9

A liquid skin care composition is prepared from the following components:

| Water Phase: | Example 6 | Example 7 | Example 8 | Example 9 |
|---|---|---|---|---|
| Water | q.s. | q.s. | q.s. | q.s. |
| Ensulizole | 1.0 | 2.0 | 1.0 | — |
| Hydroquinone | 2.0 | 1.0 | 1.5 | 0.1 |
| Triethanolamine | 0.6 | 1.1 | 0.6 | — |
| Glycerin | 7.0 | 5.0 | 20.0 | 10.0 |
| Niacinamide | 4.0 | 2.0 | 10.0 | 5.0 |
| N-Acetyl Glucosamine | 2.0 | 0.1 | 5.0 | 0.01 |
| Pentylene Glycol | 2.0 | — | 1.0 | 4.0 |
| Butylene Glycol | — | — | — | 1.0 |
| Dex-panthenol | 0.5 | 0.2 | 2.0 | 0.5 |
| Glydant Plus¹ | 0.2 | — | — | — |
| Sodium Ascorbyl | 0.02 | — | — | 0.1 |
| Phosphate | | | | |
| Disodium EDTA | 0.07 | 0.2 | 0.05 | 0.1 |
| Aloe Barbadensis Leaf | 0.1 | — | — | 0.5 |
| Juice | | | | |
| Sodium Metabisulfite | 0.15 | 0.25 | 0.15 | 0.1 |
| Benzyl Alcohol | — | 0.1 | 0.1 | — |
| Methylparaben | 0.1 | 0.1 | 0.1 | 0.1 |
| Total: | 100% | 100% | 100% | 100% |

¹DMDM hydantoin and iodopropynyl butylcarbamate from Lonza ™, Inc.

In a suitable vessel, combine all ingredients and mix until uniform. Transfer the composition into one or more suitable containers.

In one example, the compositions from examples 6 through 9 are filled into individual (break off) dose swab applicators (SwabPlus™, CA) for targeted application to specific areas of the skin, such as age spots. In an alternative example, the compositions from examples 6 through 9 are filled into small pen applicator with a porous tip (Dab-O-Matic™, NY) that dispenses the liquid compositions onto targeted areas of the skin when the pen is pressed against the skin.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a) a vitamin B3 compound;
b) a sugar amine;
c) a hydroquinone compound; and
d) a dermatologically-acceptable carrier.

2. The composition according to claim 1 wherein the composition comprises from about 0.1% to about 10% of the sugar amine.

3. The composition according to claim 2 wherein the sugar amine is N-acetyl glucosamine.

4. The composition according to claim 1 wherein the composition comprises from about 0.1% to about 10% of the vitamin B3 compound.

5. The composition according to claim 4 wherein the vitamin B3 compound is niacinamide.

6. The composition according to claim 1 wherein the composition comprises from about 0.1% to about 5% of the hydroquinone compound.

7. The composition according to claim 6, wherein the hydroquinone compound is hydroquinone.

8. The composition according to claim 1, further comprising an N-acyl amino acid.

9. The composition according to claim 8, wherein the N-acyl amino acid is undecylenoyl-L-phenylalanine.

10. The composition according to claim 1, further comprising from about 1% to about 30% of a sunscreen.

11. The composition according to claim 10, wherein the sunscreen provides a sun protection factor of from about 8 to about 45.

12. The composition according to claim 1, further comprising from about 0.01% to about 20% of a particulate material.

13. The composition according to claim 1, further comprising at least one additional skin care active.

14. A method of regulating the condition of mammalian keratinous tissue, comprising the step of applying to the keratinous tissue a personal care composition comprising a vitamin B3 compound, a sugar amine, hydroquinone, and a dermatologically-acceptable carrier.

15. The method according to claim 14, wherein the composition comprises at least one additional ingredient selected from the group consisting of an N-acyl amino acid, a sunscreen, a particulate material, at least one additional skin care active, and mixtures thereof.

16. The method according to claim 14, wherein the composition is applied with an implement, a delivery enhancement device, and combinations thereof.

17. The method according to claim 14, wherein the composition is applied to a hyperpigmented area of keratinous tissue.

18. The method according to claim 17, wherein the composition is applied with an implement selected from the group consisting of a swab, a pen, and combinations thereof.

19. A kit comprising:
a) a personal care composition comprising a vitamin B3 compound, a sugar amine, hydroquinone, and a dermatologically-acceptable carrier; and
b) an implement.

20. The kit according to claim 19 wherein the implement is a swab, a pen, and combinations thereof.

21. The kit of claim 19, further comprising at least one additional component selected from the group consisting of at least one additional personal care composition, a dietary supplement, a delivery enhancement device, a substrate, instructions for complying with an application regimen, instructions for using the implement to deliver the composition to a hyperchromic area of skin, and combinations thereof.
